(19) United States
(12) Patent Application Publication
(10) Pub. No.: US 2003/0216650 A1
(43) Pub. Date: Nov. 20, 2003

(54) Diagnosis appliance

(75) Inventor: Georg Michelson, Baiersdorf (DE)

Correspondence Address: NIXON PEABODY, LLP, 8180 GREENSBORO DRIVE, SUITE 800, MCLEAN, VA, 22102, US

(73) Assignee: e-EyeCare GmbH, Erlangen (DE)

(21) Appl. No.: 10/437,934

(22) Filed: May 15, 2003

(30) Foreign Application Priority Data
May 15, 2002 (DE) 102 21 472.7-35

(51) Int. Cl. A61B 6/00

(52) U.S. Cl. 600/476; 128/920; 382/128

(57) ABSTRACT

The invention refers to a diagnosis appliance for the diagnosis of vascular diseases of a patient. The appliance consists of: a camera to photograph des background of at least one eye of the patient; a medium for digital storage of the image of the eye background taken with the camera; a medium for reading the stored image and a medium for determining specified parameters by evaluation of the stored image. This makes a quick and cost-saving examination possible to identify stroke risk endangered patients.

### BACKGROUND OF THE INVENTION

1. Field of the Invention

The invention refers to a diagnosis appliance for the diagnosis of the vascular diseases of a patient.

Patients, suffering from a stroke, usually have had, long before the actual stroke, a hypertonus of the arteries. There is a number of other known risk factors in addition to that increasing the stroke risk (i.e. overweight etc.). In spite of that, it has been very difficult so far to conduct a precise diagnosis without large-scale examinations which finally give the information about how high the actual danger of a stroke is.

2. Description of Related Art

An improved diagnosis procedure for the evaluation of the stroke risk was described by L. D. Hubbard et al. in “Methods For Evaluation Of Retinal Microvascular Abnormalities Associated With Hypertension/Sclerosis In The Atherosclerosis Risk In communities Study”, Ophthalmology, Vol. 106, 2269-2280, 1999. Here, the eye background and namely the retina are looked at very closely. The vessels of the retina and those of the brain are quite similar. Both vessels show early changes due to which a conclusion in regard to an increased stroke risk can be made. Long-term hypertension changes the vessels in the brain and on the retina in a characteristic and measurable way. Therefore, the vessels of the retina are visible indicators for vessel damages in the brain since the retina is part of the brain.

Within the scope of the a.m. diagnosis method, the relationship between the sum of all arterial vessel diameters and the sum of all vein diameters (a-v-ration) is determined. The quotient supplies a reliable criterion for an evaluation of the stroke risk.

It would prevent many people from suffering and above that would reduce the costs for healthcare essentially if it became possible to carry out the said examination on a larger scale and in this especially simple way which would make possible an easy and precise diagnosis of the stroke risk. If necessary, the patients inflicted could be identified early and therapy could be started before a stroke happened. Early discovery of the risk and an early start therapy considerably reduce the danger of a stroke.

Precondition for this is a simple method of examination and the necessary technical equipment to carry out such an examination on a large scale and at moderate costs. This is, where the disadvantages of the above described methods are: the examination itself is simple, yet, in order to get most out of it, a large number of patients needs to be examined to identify the risk patients. So far, known diagnosis methods have not been suitable for carrying out the necessary number of examinations quickly and at moderate costs.

The invention is based on the CHALLENGE to create a diagnosis appliance which makes it possible in a very simple way to obtain and evaluate the necessary data in order to be able to examine a great number of patients in an efficient and cost-saving way. The appliance should especially make it possible to easily supply the information necessary for the diagnosis of the participating patients and otherwise create the technical preconditions for the evaluation of the data. The diagnosis appliance is easily set up and allows a quick and simple examination. It is supposed to offer the pre-requisite for a cost-saving examination of a great number of patients and the quick identification of endangered patients.

The invention SOLVES the problem with a diagnosis appliance with the following features:

a camera to take pictures of the eye background of at least one eye of the patient;

a medium for the digital storage of the eye background pictures taking by the camera;

a medium to read the stored image and

a medium to determine specified parameters by evaluating the stored image.

For a precise picture of the eye background, the appliance must have a fixture to hold and position the patient's head.

The camera is connected to an image processing system which transforms the image of the eye background taken by the camera into a digitally storable format. The image processing system is connected via data communication equipment with the medium for the digital storage o the eye background image. It is also possible to connect the storage medium of the image with the reading medium of the stored image via data communication equipment.

Preferably, the camera for taking the eye background picture on the one hand, and the digital medium for storing the image on the other and finally the reading medium with the analyzer should be placed at three different locations at great distances from each other, all communicating via a data communicating equipment. As the communicative means, the data communication equipment used here is the internet.

The camera for taking the eye background pictures should preferably be a high solution digital camera. A Non-Mydriatic Fundus camera which in it special simplicity is well suited for the pictures of the eye background.

It is important to secure that only authorized personnel have access to the system or the stored data. A training program will secure the means to rule out unauthorized access to especially the digital storage.

The evaluation media need to be suitable for the calculation of the quotient of all sums of the arterial vessel diameters and the sum of all diameters of the veins at least of a chosen section of the photographed eye background. An image processing software can be used for this which should be thus that the evaluation media for automatic image recognition and evaluation are suitable to recognize arterial and vein vessel and determine their diameters.

For an efficient data management, a further training program will see to it that the digital storage media of the eye background image taken with the camera are suitable to add to the memory area of the image another memory area with the medial evidence. The medical evidence can be fed in via input media into the memory area for the evidence.

The invention creates a diagnosis appliance which can automatically carry out the calculation of the relations of the sum of all arterial vessel diameters and the sum of all vein vessel diameters, the a-v-ratio, from the retinal fundus images. The diagnosing doctor's work is considerably easier. The information transfer through data communication from the examination location (camera location) to the location of evaluation (location of evaluation media) creates the necessary flexibility in order to examine a large number of patients in short time.

It is possible to record eye background images of a large number of patients at specially made appointments and later on another location (the evaluation center) synergetically analyze the data. An economical identification of patients with a stroke risk becomes possible.

The suggested diagnosis is appliance allows an institution encroaching communication based on evidence for the early identification of stroke risk patients due to their retina vessels and electronical making available of the necessary information for authorized doctors and patients.

### BRIEF DESCRIPTION OF THE DRAWING

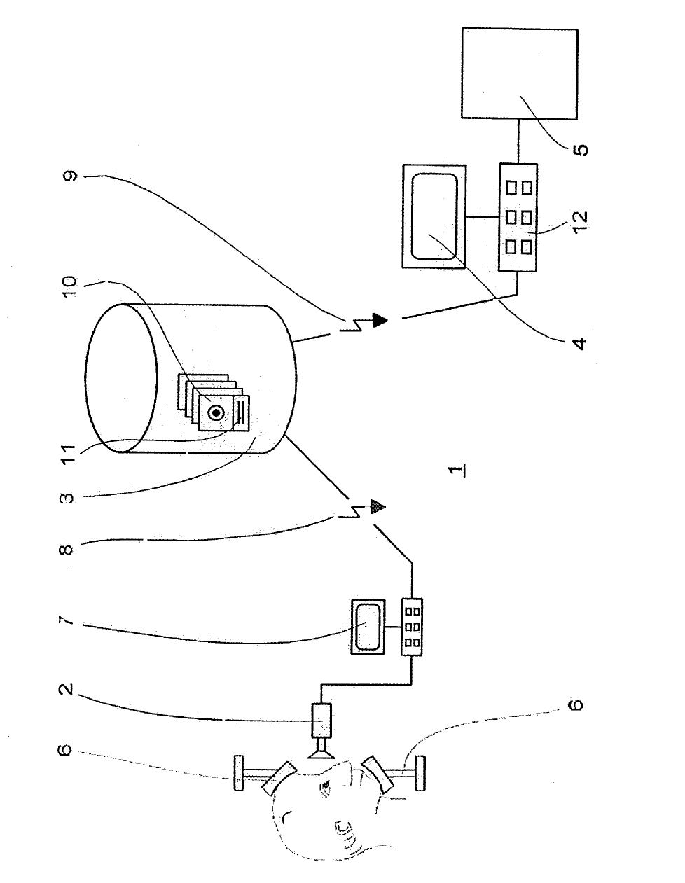

The drawing (FIG. 1) shows a sample of the invention showing very schematically the complete diagnosis appliance for the identification of stroke risk patients.

### DETAILED DESCRIPTION OF THE INVENTION

The entire diagnosis appliance is marked 1. Showing—on the examination location of the patients—a fixture 6 for the patient's head. The head is placed in the fixture 6 and kept still. Then the background of the patient's eye is photographed with a Non-Mydriatic Fundus camera 2. The camera 2 is connected with an image processing system 7 which transforms the photographed image into a digitally storable format, if the camera 2 itself does not directly supply a digital image. Via a secured data communication equipment 8, very schematized, the digital image of the eye background is brought to a digital memory 3 and deposited there. IN the digital memory 3 is a great number of files, each file representing a patient file. The file of a patient has a memory area 10 for the digitalized image of the eye background. Then, a.o., there is a memory area 11 where the medical evidence can be stored.

The digital memory media 3 are connected via data communication equipment 9, again very schematized, with a reading medium 4 which makes the image of the eye background visible. In the simplest case, a monitor. The reading medium 4 is connected with an evaluation medium 5 which is able via a suitable and common image evaluation software to carry out a graphic analysis of the picture of the eye background. Here via suitable digital processes in a given section of the picture (resp. the cross-section visible on the picture) the determination of the diameter of the arterial vessels of the retina on the one hand and of the vein vessels on the other hand are carried out. The evaluation media ad up the sums of the determined volumina (resp. areas), then get the quotients of the sums. After an automatical segmentation of the retinal arteries and veins and an automatical determination of the diameters of all vessels larger than 30 μm said quotient can be calculated.

This quotient—the a-v-ratio—from the retinal Fundus image supplies, according to L. D. Hubbard (s.a.), a criteria for the evaluation of the stroke risk the patient is exposed to. An evaluation in figures regarding the condition of the eye background vessels in regard to vessel damage through hypertonus.

After automatically obtaining the a-v-ratio through the evaluation media 5, the doctor can make his diagnosis and deposit it via input media 12 and the data communication equipment 9 in the memory area 11.

The process of an examination could be as follows—

First, the public is informed about the examination method and if interested, invited to the examination locations. On the location, the necessary personal data of the patient are noted in order to identify he or she and also—for a later diagnosis—be able to supply the doctor with the information necessary for a diagnosis (age, gender, weight, height etc.). Insurance data are obtained as well, for instance by reading the insurance chip card through a reading device. After a short anamnesis, the eye background is photographed with the camera 2. This can be done by a medical assistant without eye drops and Mydriasis. The photograph of the eye background takes place totally without contact so that there is no strain on the patient. Via image processor 7, the image data together with patient data are transferred to memory medium 3.

At the evaluation center, the diagnosing doctor calls up the case, i.e. the stored data, via reading medium 4 from the memory medium 3. The evaluation media 5 carry out an evaluation of the Fundus image with a special image processing software and calculate the a.m. a-v-ratio (quotient). The doctor can now make a diagnosis and store it via input 12 in the memory area 11 of memory medium 3.

Via a (secured) web access with a password it is then possible, from any location, to have access to memory medium 3 and a certain patient case and inform the patient about the results.

For data protection the usual and common elements are used. Every doctor is identified with a chip card with a certain characteristic. The data movement happens secured and traceable because any transaction is protocoled and therefore the authenticity of the medical evidence is verifiable at any time. The data transfer is secured in such a way as the data between the individual stations (between image processing system 7 and memory medium 3 resp. between memory medium 3 and reading medium 4) are transferred encoded (128 bit code). Sensitive data can deposited in memory medium 3 (server) encoded. The coding prevents the storage of personal data. For data securing, the storage will always be carried out on two hard disks at the same time. The server (memory medium 3) can be physically secured by an electronic lock system within the security computer center. The server can be secured with a firewall.

Note: the entire examination including diagnosis take place mainly on the basis of physically measurable parameters (quantitative analysis of the vessel changes); for the evaluation of the stroke risk, specified quotients are calculated, with the help of the evaluation of the image data (determination of the arterial and vein vessel diameters larger than 30 μm, summing up, quotient obtaining). Described examination could be carried out by persons without medical training, taking care of the data obtaining and the automatical evaluation of the data in the evaluation medium. Finally, the entire examination can be carried out pretty much automized which would increase the efficiency of the system even more.

The possibility of a remote diagnosis (on location of the ready 4 and evaluation 5 media)—with or without a doctor—can be an important instrument to make a quick and cost-saving examination available to a great number of patients.

What is claimed is:

1. Diagnosis appliance for the diagnosis of vascular diseases of a patient consisting of
a camera for photographing the background of at least one eye of the patient;
A medium for the digital storage of the image of the eye background taken with the camera
a medium for reading the stored image and
a medium for determining the specified parameters by evaluation of the stored image.

2. Diagnosis appliance according to claim 1, characterized by a fixture for holding and positioning the patient's head.

3. Diagnosis appliance according to claim 1 or 2, characterized by the fact that the camera is connected with an image processing system, which transforms the image of the eye background taken by the camera into a digitally storable format.

4. Diagnosis appliance according to claim 3, characterized by the fact that an image processing system is connected with a medium for digital storage of the image of the eye background via a data communication equipment.

5. Diagnosis appliance according to one of the claims 1 to 4, characterized by the fact that the medium for digital storage of the image of the eye background is connected with the medium for reading the stored image via a data communication equipment.

6. Diagnosis appliance according to one of the claims 1 to 5, characterized by the fact that the camera for photographing the eye background on the one hand, the medium for digital storage of the picture taken with the camera on the other hand and finally the media for reading of the stored image and for determining the specified parameters by evaluating the stored image, are placed on three different locations at a great distance form each other, each connected via data communication equipment.

7. Diagnosis appliance according to one of the claims 4 to 6, characterized by the fact that the data communication equipment is the internet.

8. Diagnosis appliance according to one of the claims 1 to 7, characterized by the fact that the camera for photographing the eye background is a high solution digital camera.

9. Diagnosis appliance according to one of the claims 1 to 8, characterized by the fact that the camera for photographing the eye background is a Non-Mydriacic Fundus camera.

10. Diagnosis appliance according to one of the claims 1 to 9, characterized by the fact that there are secure means to prevent unauthorized access to the media for digital storage.

11. Diagnosis appliances according to one of the claims 1 to 10, characterized by the fact that the media for determining specified parameters for the calculation of the quotient of the sum of all arterial vessel diameters and the sum of all vein vessel diameters are suitable at least for a section of the photographed eye background.

12. Diagnosis appliance according to claim 11, characterized by the fact that the media for determining specified parameters for automatical image recognition and evaluation are suitable to identify arterial and vein vessel and determine their diameters.

13. Diagnosis appliance according to one of the claims 1 to 12, characterized by the fact that the media for digital storage of the image of the eye background taken by the camera are suitable to add to the memory area of the image a memory area consisting of the medical evidence.

14. Diagnosis appliance according to claim 13, characterized by the fact that the medical evidence can be deposited in the memory area for the medical evidence via input medium.
